(19) United States
(12) Patent Application Publication
(10) Pub. No.: US 2017/0326369 A1
(43) Pub. Date: Nov. 16, 2017

(54) TRANS SEPTAL IMPLANTABLE MEDICAL DEVICE

(71) Applicant: CARDIAC PACEMAKERS, INC., St. Paul, MN (US)

(72) Inventors: Brendan Early Koop, Ham Lake, MN (US); Benjamin J. Haasl, Forest Lake, MN (US); Angelo Auricchio, Magdeburg (DE); Brandon Christopher Fellows, Minneapolis, MN (US)

(73) Assignee: CARDIAC PACEMAKERS, INC., St. Paul, MN (US)

(21) Appl. No.: 15/589,606

(22) Filed: May 8, 2017

Related U.S. Application Data
(60) Provisional application No. 62/334,182, filed on May 10, 2016.

(51) Int. Cl. A61N 1/368; A61N 1/05; A61N 1/375; A61N 1/378

(52) U.S. Cl. CPC A61N 1/3684; A61N 1/3787; A61N 1/0573; A61N 1/3756

(57) ABSTRACT

An implantable medical device may include a housing configured to be positioned at least in part in a right ventricle (RV) proximate an RV facing side of the ventricular septum. A power source and circuitry may be disposed within the housing and may be operatively coupled together. An RV electrode may be fixed relative to the housing to be proximate the RV facing side of the ventricular septum and may be operatively coupled with the circuitry. An LV electrode support may extend away from the housing into the ventricular septum toward the LV facing side of the ventricular septum and may support an LV electrode that is operatively coupled with the circuitry. The circuitry may be configured to pace the RV of the patient's heart using the RV electrode and to pace the LV of the patient's heart using the LV electrode.

### CROSS REFERENCE TO RELATED APPLICATIONS

This application claims the benefit of U.S. Provisional Patent Application Ser. No. 62/334,182 filed on May 10, 2016, the disclosure of which is incorporated herein by reference.

### TECHNICAL FIELD

The disclosure relates generally to implantable medical devices, and relates more particularly to implantable medical devices that may extend at least partially through a septum within a patient's heart.

### BACKGROUND

Implantable medical devices are commonly used today to monitor a patient and/or deliver therapy to a patient. For example, implantable sensors are often used to monitor one or more physiological parameters of a patient, such as heart beats, heart sounds, ECG, respiration, etc. In another example, pacing devices are often used to treat patients suffering from various heart conditions that may result in a reduced ability of the heart to deliver sufficient amounts of blood to a patient's body. Such heart conditions may lead to slow, rapid, irregular, and/or inefficient heart contractions. To help alleviate some of these conditions, various medical devices (e.g., pacemakers, defibrillators, etc.) can be implanted in a patient's body. Such devices may monitor and in some cases provide electrical stimulation to the heart to help the heart operate in a more normal, efficient and/or safe manner. Under some circumstances, it can be beneficial to sense and/or pace two or more chambers of the heart.

### SUMMARY

This disclosure relates generally to implantable medical devices, and relates more particularly to implantable medical devices that extend at least partially through a septum within a patient's heart. In some cases, the implantable medical devices may be configured to be disposed adjacent a septum within a patient's heart. In some cases, the implantable medical devices may be configured to extend at least partially into the septum, and in some cases, entirely through the septum. The septum may be the ventricular septum the divides the left and right ventricles. When so provided, the implantable medical device may be configured to sense and/or pace both ventricles of the heart. In some cases, the implantable medical device may be delivered to the ventricle septum via the more accessible right ventricle of the heart.

In one example of the disclosure, an implantable medical device (IMD) is configured for deployment at a ventricular septum of a patient's heart, the ventricular septum of the patient's heart having a right ventricle (RV) facing side and a left ventricle (LV) facing side. The IMD may include a housing configured to be positioned at least in part in the right ventricle (RV) of the patient's heart proximate the RV facing side of the ventricular septum once the IMD is implanted in the patient's heart. The IMD may further include a power source that is disposed within the housing. Circuitry may be disposed within the housing and may be operatively coupled to the power source. A first RV electrode may be fixed relative to the housing and may be positioned to be proximate the RV facing side of the ventricular septum once the IMD is implanted. The first RV electrode may be operatively coupled with the circuitry in the housing. A second RV electrode may be fixed relative to the housing and spaced from the first RV electrode. The second RV electrode may be operatively coupled with the circuitry in the housing. An LV electrode support may extend away from the housing into the ventricular septum toward the LV facing side of the ventricular septum. An LV electrode may be supported by the LV electrode support and may be spaced from the housing. The LV electrode may be operatively coupled with the circuitry in the housing. The circuitry may be configured to pace the RV of the patient's heart using the first RV electrode and/or the second RV electrode and the LV of the patient's heart using the LV electrode.

Alternatively or additionally, the first RV electrode and/or the second RV electrode may be disposed on an outer surface of the housing.

Alternatively or additionally to any of the embodiments above, the LV electrode support and the LV electrode may be configured to position the LV electrode within the ventricular septum.

Alternatively or additionally to any of the embodiments above, the LV electrode support and the LV electrode may be configured to position the LV electrode in contact with the LV facing side of the ventricular septum.

Alternatively or additionally to any of the embodiments above, the IMD may further include another LV electrode support extending away from the housing into the ventricular septum toward the LV facing side of the ventricular septum, and another LV electrode supported by the another LV electrode support. The another LV electrode may also be operatively coupled with the circuitry in the housing.

Alternatively or additionally to any of the embodiments above, the LV electrode support may be configured to extend far enough into the ventricular septum to position the LV electrode for successful capture of the LV of the patient's heart, while not extending all the way through the ventricular septum.

Alternatively or additionally to any of the embodiments above, the IMD may be a dual chamber leadless cardiac pacemaker (LCP).

Alternatively or additionally to any of the embodiments above, the power source may comprise a battery.

Alternatively or additionally to any of the embodiments above, the power supply may comprise a rechargeable power source, such as a rechargeable battery, a capacitor such as a super-capacitor and/or any other suitable rechargeable power source.

In another example of the disclosure, an implantable medical device (IMD) may be configured for deployment at a ventricular septum of a patient's heart. The IMD may include a housing that is configured to be positioned at least in part in the right ventricle of the patient's heart proximate the right ventricle facing side of the ventricular septum once the IMD is implanted. The IMD may also include a power source disposed within the housing. Circuitry may be disposed within the housing and may be operatively coupled to the power source. An RV electrode may be fixed relative to the housing and positioned to engage the right ventricle facing side of the ventricular septum once the IMD is implanted. The RV electrode may be operatively coupled with the circuitry. An LV electrode assembly may include a septal portion configured to extend away from the housing and through the ventricular septum and a hook portion that extends distally from the septal portion and is configured to engage the LV facing side of the ventricular septum once the IMD is implanted. An LV electrode may be supported by the hook portion to engage the left ventricle facing side of the ventricular septum once the IMD is implanted. The circuitry may be configured to pace the RV of the patient's heart using the RV electrode and the LV of the patient's heart using the LV electrode.

Alternatively or additionally to any of the embodiments above, the LV electrode assembly may extend distally from a distal end of the housing and may have an effective length sufficient to place the first hook portion in contact with the LV facing side of the ventricular septum.

Alternatively or additionally to any of the embodiments above, the septal portion of the LV electrode assembly may have a length of up to about 2 centimeters.

Alternatively or additionally to any of the embodiments above, the LV electrode assembly may be configured to anchor the IMD in place relative to the ventricular septum once the IMD is implanted.

Alternatively or additionally to any of the embodiments above, the septal portion of the LV electrode assembly may include an electrically insulating outer surface.

Alternatively or additionally to any of the embodiments above, the hook portion of the LV electrode assembly may be movable between a substantially linear configuration for insertion through the ventricular septum and a curved configuration in which the hook portion positions the LV electrode in contact with the LV facing side of the ventricular septum.

Alternatively or additionally to any of the embodiments above, the circuitry may be further configured to sense cardiac electrical activity using the RV electrode and the LV electrode.

Alternatively or additionally to any of the embodiments above, the power source may include a battery.

Alternatively or additionally to any of the embodiments above, the power supply may comprise a rechargeable power source, such as a rechargeable battery, a capacitor such as a super-capacitor and/or any other suitable rechargeable power source.

In another example of the disclosure, an implantable medical device (IMD) may be configured for deployment at a ventricular septum of a patient's heart. The IMD may include a housing that is configured to be positioned at least in part in the right ventricle of the patient's heart proximate the right ventricle facing side of the ventricular septum once the IMD is implanted. The IMD may also include a power source disposed within the housing. Circuitry may be disposed within the housing and may be operatively coupled to the power source. An RV electrode may be fixed relative to the main housing and may be positioned to engage the right ventricle facing side of the ventricular septum once the IMD is implanted. The RV electrode may be operatively coupled with the circuitry. An LV electrode support may be configured to extend from the housing into the ventricular septum, and an LV electrode may be supported by the LV electrode support. The LV electrode may be operatively coupled with the circuitry. The circuitry may be configured to pace the patient's heart using the RV electrode and the LV electrode.

Alternatively or additionally to any of the embodiments above, the LV electrode support may be configured to extend far enough into the ventricular septum to position the LV electrode for successful capture of the LV of the patient's heart, while not extending all the way through the ventricular septum.

The above summary is not intended to describe each and every disclosed embodiment or every implementation of the present disclosure. The Figures and Description which follow more particularly exemplify these and other illustrative embodiments.

### BRIEF DESCRIPTION OF THE FIGURES

The disclosure may be more completely understood in consideration of the following description in connection with the accompanying drawings, in which:

FIG. 1 is a schematic illustration of a human heart;

FIG. 2 is a schematic diagram of an illustrative implantable medical device (IMD) that may be disposed relative to the ventricular septum;

FIG. 3 is a schematic diagram of an illustrative IMD that may be disposed relative to the ventricular septum;

FIG. 4 is a schematic diagram of an illustrative IMD disposed proximate the ventricular septum;

FIG. 5 is a schematic diagram of an illustrative IMD disposed proximate the ventricular septum;

FIG. 6 is a schematic diagram of a first illustrative IMD disposed proximate the atrial septum and a second illustrative IMD disposed proximate the ventricular septum; and

FIG. 7 is a schematic block diagram of an illustrative leadless cardiac pacemaker (LCP), which may be considered as being an example of one of the IMDs of FIGS. 2 through 5.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following description should be read with reference to the drawings in which similar structures in different drawings are numbered the same. The drawings, which are not necessarily to scale, depict illustrative embodiments and are not intended to limit the scope of the disclosure.

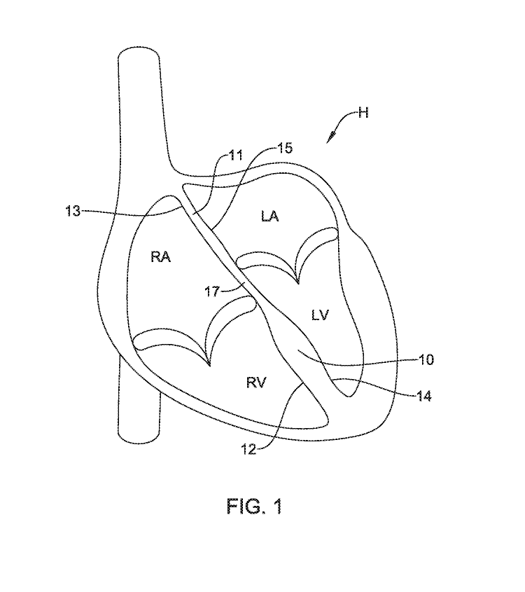

FIG. 1 is a schematic illustration of a human heart H. The heart H includes a right side and a left side, relative to the person's perspective. The right side of the heart H includes an RA (right atrium) and an RV (right ventricle). The left side of the heart H includes an LA (left atrium) and an LV (left ventricle). A ventricular septum 10 separates the RV and the LV and an atrial septum 11 separates RA and the LA. The heart H may also be considered as including an atrioventricular septum 17 between the RA and the LV. The ventricular septum 10 may be considered as having an RV facing side 12 and an LV facing side 14. The atrial septum 11 may be considered as including a RA facing side 13 and a RA facing side 15.

It is known that portions of the heart wall, including the ventricular septum 10, have conduction pathways that are involved in causing contractions in the RV and the LV. In some cases, reaching the RV through the vasculature, such as through the superior vena cava or the inferior vena cava and through the right atrium (not illustrated), may be easier than reaching the LV in an intravascular approach. In some cases, debris may be formed within the heart H as a result of placing and manipulating implantable devices within the heart H. In some cases, debris within the RV may be less problematic for the patient than debris within the LV, as debris within the RV may pass into the patient's lungs which can act as a filter while potential debris within the LV may pass directly into the patient's brain, potentially causing a stroke or other complications. Moreover, in some cases, the presence of a significant foreign object (e.g. an implantable medical device) within the heart H may cause tissue ingrowth and/or clotting to occur as a result of the body's natural response to the presence of the foreign body. Such clots, if released, are less of a concern in the RV than the LV.

In some instances, an Implantable Medical Device (IMD) may be configured to be deployed within the RV, next to or proximate the RV facing side 12 of the ventricular septum 10. In some cases, as will be discussed, an IMD may additionally or alternatively be deployed within the RA, next to or proximate the RA facing side 13 of the atrial septum 11. In some cases, a portion of the IMD may, for example, extend partially into the ventricular septum 10, or even completely through the ventricular septum 10, in order to place one or more electrodes in position to capture the aforementioned conduction pathways through the ventricular septum 10 that control the contraction of the LV, or to otherwise sense or pace within the LV. It will be appreciated that in some cases, the portion or portions of the IMD that penetrate into the LV may be minimized in size in order to minimize the body's natural response to such a foreign body. In some cases, the portion or portions of the IMD that penetrate into the LV, and in some instances even the portion or portions of the IMD that remain within the RV, may be coated with or otherwise include one or more anticoagulant materials or materials that encourage endothelialization of the surfaces exposed to blood flow in the chamber.

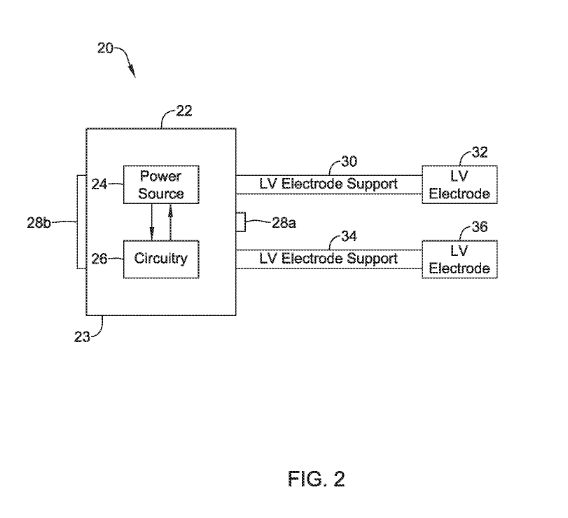

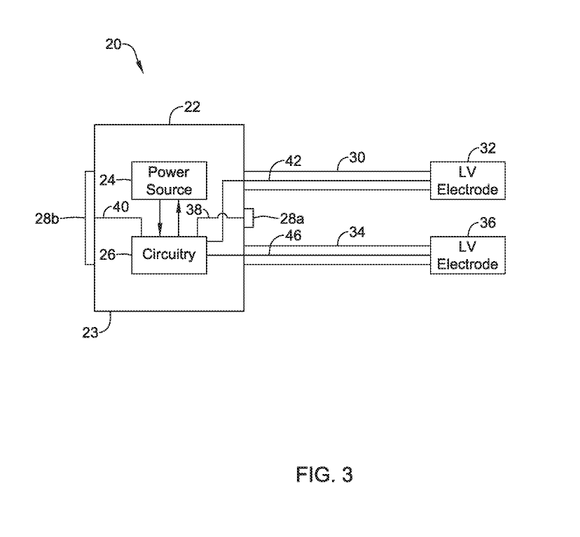

FIGS. 2 and 3 are highly schematic diagrams of an illustrative IMD 20 that may be utilized proximate the RV facing side 12 of the ventricular septum 10. In some cases, the IMD 20 may include a housing 22 that is configured to be disposed at least partially within the RV, next to or proximate the RV facing side 12 of the ventricular septum 10. A power source 24 may be disposed within the housing 22. In some cases, the power source 24 may be a battery. In some instances, the power source 24 may be a rechargeable power source, such as a rechargeable battery, a capacitor such as a super-capacitor and/or any other suitable rechargeable power source or source capable of harvesting power transmitted wirelessly from another device. In some cases, for example, energy may be transmitted to the power source 24 via RF power transfer. Circuitry 26 may be disposed within the housing 22 and may be operatively coupled to the power source 24 such that the power source 24 can power operation of the circuitry 26. In some cases, if the power source 24 is rechargeable, the circuitry 26 may also regulate recharging operations of the power source 24. In some cases, the circuitry 26 may include or be coupled to an antenna, inductive loop and/or other energy receiving element for wirelessly receiving energy to recharge the battery.

A first RV electrode 28a and a second RV electrode 28b may be fixed relative to the housing 22 and in some cases at least one of the first RV electrode 28a and the second RV electrode 28b may be positioned relative to the housing 22 in order to engage the RV facing side 12 of the ventricular septum 10 once the IMD 20 has been implanted. In some instances, the first RV electrode 28a may be relatively smaller than the second RV electrode 28b. The first RV electrode 28a may, for example, function as a cathode electrode while the second RV electrode 28b may function as an anode electrode. In some cases, one or more of the first RV electrode 28a and/or the second RV electrode 28b may be disposed on an outer surface 23 of the housing 22, and in some instances on an end of the housing 22. In some cases, as seen in FIG. 3, the first RV electrode 28a and the second RV electrode 28b may be operably coupled with the circuitry 26 via communication lines 38 and 40, respectively.

In some cases, the IMD 20 may include an LV electrode support 30 that extends away from the housing 22 and at least partially into the ventricular septum 10 towards the LV facing side 14 of the ventricular septum 10. In some instances, an LV electrode 32 may be supported by the LV electrode support 30 and may be operably coupled with the circuitry 26. In some instances, the LV electrode support 30 may be configured to extend far enough into the ventricular septum 10 to position the LV electrode 32 for successful capture of the LV of the heart H without extending all of the way through the ventricular septum 10.

In some cases, the LV electrode support 30 and the LV electrode 32 are configured to position the LV electrode 32 within the ventricular septum 10. In some cases, the LV electrode support 30 and the LV electrode 32 are configured to position the LV electrode 32 in contact with the LV facing side 14 of the ventricular septum 10. In some cases, as seen in FIG. 3, the LV electrode 32 may be operably coupled to the circuitry 26 via a communication line 42, extending through the LV electrode support 30.

In some cases, another LV electrode support 34 may extend away from the housing 22 and may be configured to extend at least partially into the ventricular septum 10 towards the LV facing side 14 of the ventricular septum 10, although in some cases the IMD 20 may not include the LV electrode support 34. In some instances, another LV electrode 36 may be supported by the LV electrode support 34 and may be operably coupled with the circuitry 26 via a communication line 46, which extends through the LV electrode support 34 as seen in FIG. 3. In some cases, the circuitry 26 may be configured to sense and/or pace the RV of the heart H using the first RV electrode 28a and/or the second RV electrode 28b and to sense and/or pace the LV of the heart H using the LV electrode 32.

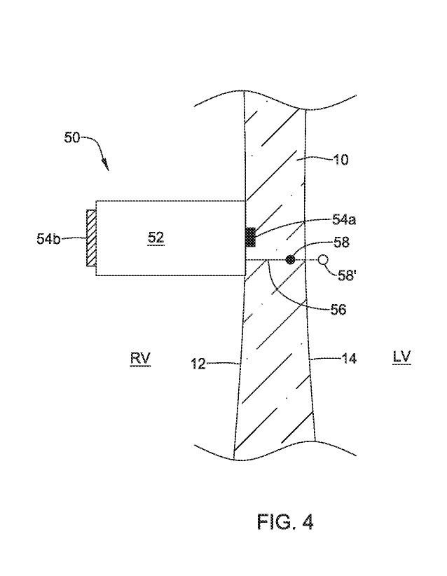

FIG. 4 is a schematic diagram of an illustrative IMD 50 that is positioned within the RV, adjacent the RV facing side 12 of the ventricular septum 10. The illustrative IMD 50 includes a housing 52 that may, for example, house internal components such as but not limited to those described with respect to the IMD 20 (FIGS. 2 and 3). In some cases, the IMD 50 includes a first RV electrode 54a and/or a second RV electrode 54b that may each be fixed relative to the housing 52 to place at least one of the first RV electrode 54a and/or the second RV electrode 54b proximate and/or in contact with the RV facing side 12 of the ventricular septum 10 when the IMD 50 is implanted within the heart H. In some cases, the first RV electrode 54a may function as a cathode electrode while the second RV electrode 54b may function as an anode electrode. The cathode electrode may have a significantly smaller surface area than the anode electrode.

In some cases, an LV electrode support 56 may extend from the housing 52 and at least partially through the ventricular septum 10. An LV electrode 58 may be secured to or otherwise supported by the LV electrode support 56. In some cases, and for some patients, the LV electrode support 56 is configured to place the LV electrode 58 within the ventricular septum 10, and in electrical communication with conduction pathways extending through the ventricular septum 10 that control the contraction of the LV. In some instances, the LV electrode support 56 may extend entirely through the ventricular septum 10 in order to place an LV electrode 58′, shown in phantom, within the LV and in contact with the LV facing side 14 of the ventricular septum 10.

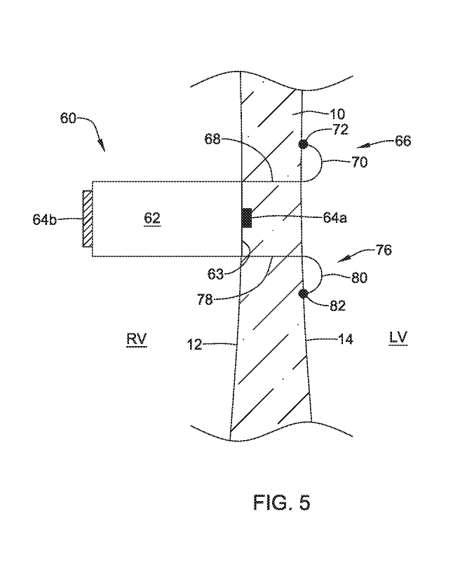

FIG. 5 is a schematic diagram of another illustrative IMD 60 that is positioned within the RV, adjacent the RV facing side 12 of the ventricular septum 10. The illustrative IMD 60 includes a housing 62 that houses internal components such as but not limited to those described with respect to the IMD 20 (FIGS. 2 and 3). In some cases, the IMD 60 includes a first RV electrode 64a and a second RV electrode 64b that may each be fixed relative to the housing 62 so as to place at least one of the first RV electrode 64a and/or the second RV electrode 64b proximate and/or in contact with the RV facing side 12 of the ventricular septum 10 when the IMD 60 is implanted with the heart H. In some cases, the first RV electrode 64a may function as a cathode electrode while the second RV electrode 64b may function as an anode electrode.

In some cases, the IMD 60 may include an LV electrode assembly 66 that may, for example, be configured to anchor the IMD 60 in place relative to the ventricular septum 10. In some cases, the LV electrode assembly 66 includes a septal portion 68 that is configured to extend away from the housing 62 and through the ventricular septum 10, as well as a hook portion 70 that extends distally from the septal portion 68 and hooks around to engage the LV facing side 14 of the ventricular septum 10 when the IMD 60 is implanted in the heart H, as illustrated. In some cases, the septal portion 68 may include an electrically insulating outer surface. In some instances, the hook portion 70 may be movable between a substantially linear configuration for insertion through the ventricular septum 10 and a curved or hooked configuration (as shown) in which the hook portion 70 positions an LV electrode 72 in contact with the LV facing side 14 of the ventricular septum 10. The LV electrode 72 may be electrically connected to control circuitry housed by the housing 62 by one or more conductors extending along the hook portion 70 and septal portion 68. The hook portion 70 may also function to anchor the IMD 60 to the ventricular septum 10.

In some cases, the septal portion 68 of the LV electrode assembly 66 extends distally from a distal end 63 of the housing 62 and has an effective length that is sufficient to place the hook portion 70 in the LV and in contact with the LV facing side 14 of the ventricular septum 10. In one example, the septal portion 68 may have an effective length of about 2 centimeters or less. In some cases, the LV electrode 72 may be supported by the hook portion 70 and may be configured to engage the LV facing side 14 of the ventricular septum 10 as shown. It will be appreciated that the IMD 60 may include circuitry, such as the circuitry 26 (FIGS. 2 and 3), that enable the IMD 60 to sense and/or pace the RV of the heart H using one or more of the first RV electrode 64a and/or the second RV electrode 64b and to sense and/or pace the LV of the heart H using the LV electrode 72.

In some cases, the IMD 60 may include another LV electrode assembly 76 that may, for example, also be configured to anchor the IMD 60 in place relative to the ventricular septum 10. In some cases, the LV electrode assembly 76 may include a septal portion 78 that is configured to extend away from the housing 62 and through the ventricular septum 10, as well as a hook portion 80 that extends distally from the septal portion 78 and hooks around to engage the LV facing side 14 of the ventricular septum 10 when the IMD 60 is implanted in the heart H, as illustrated. In some cases, the septal portion 78 may include an electrically insulating outer surface. In some instances, the hook portion 80 may be movable between a substantially linear configuration for insertion through the ventricular septum 10 and a curved or hooked configuration (as shown) in which the hook portion 80 positions an LV electrode 82 in contact with the LV facing side 14 of the ventricular septum 10. The LV electrode 82 may be electrically connected to control circuitry housed by the housing 62 by one or more conductors extending along the hook portion 80 and septal portion 78. The hook portion 80 may also function to anchor the IMD 60 to the ventricular septum 10.

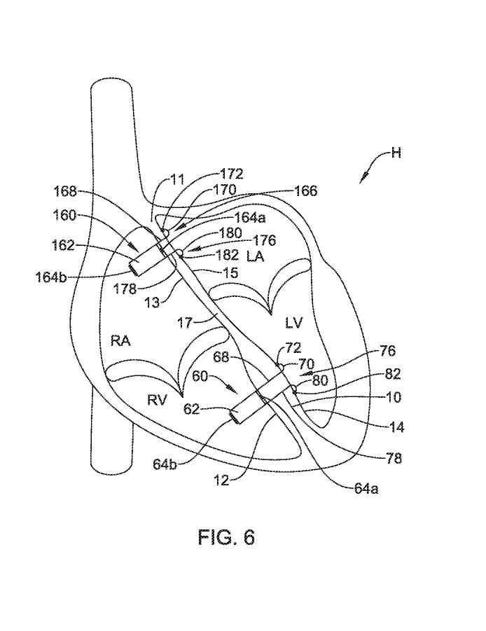

FIG. 6 is a schematic diagram of the heart H, showing the IMD 60 disposed within the RV as well as an IMD 160 that is disposed within the RA. In some cases, the illustrative IMD 160 includes a housing 162 that houses internal components such as but not limited to those described with respect to the IMD 20 (FIGS. 2 and 3). In some cases, the IMD 160 includes a first RA electrode 164a and a second RA electrode 164b that may each be fixed relative to the housing 162 so as to place at least one of the first RA electrode 164a and/or the second RA electrode 164b proximate and/or in contact with the RA facing side 13 of the atrial septum 11 when the IMD 160 is implanted with the heart H. In some cases, the first RA electrode 164a may function as a cathode electrode while the second RA electrode 164b may function as an anode electrode.

In some cases, the IMD 160 may include an LA electrode assembly 166 that may, for example, be configured to anchor the IMD 160 in place relative to the atrial septum 11. In some cases, the LA electrode assembly 166 includes a septal portion 168 that is configured to extend away from the housing 162 and through the atrial septum 11, as well as a hook portion 170 that extends distally from the septal portion 168 and hooks around to engage the LA facing side 15 of the atrial septum 11 when the IMD 160 is implanted in the heart H, as illustrated. In some cases, the septal portion 168 may include an electrically insulating outer surface. In some instances, the hook portion 170 may be movable between a substantially linear configuration for insertion through the atrial septum 11 and a curved or hooked configuration (as shown) in which the hook portion 170 positions an LA electrode 172 in contact with the LA facing side 15 of the atrial septum 11. The LA electrode 172 may be electrically connected to control circuitry housed by the housing 162 by one or more conductors extending along the hook portion 170 and the septal portion 168. The hook portion 170 may also function to anchor the IMD 160 to the atrial septum 11.

In some cases, the septal portion 168 of the LA electrode assembly 166 extends distally from the housing 162 and has an effective length that is sufficient to place the hook portion 170 in the LA and in contact with the LA facing side 15 of the atrial septum 11. In some cases, the LA electrode 172 may be supported by the hook portion 170 and may be configured to engage the LA facing side 15 of the atrial septum 11 as shown. It will be appreciated that the IMD 160 may include circuitry, such as the circuitry 26 (FIGS. 2 and 3), that enable the IMD 160 to sense and/or pace the RA of the heart H using one or more of the first RA electrode 164a and/or the second RA electrode 164b and to sense and/or pace the LA of the heart H using the LV electrode 172.

In some cases, the IMD 160 may include another LA electrode assembly 176 that may, for example, also be configured to anchor the IMD 160 in place relative to the atrial septum 11. In some cases, the LA electrode assembly 176 may include a septal portion 178 that is configured to extend away from the housing 162 and through the atrial septum 11, as well as a hook portion 180 that extends distally from the septal portion 178 and hooks around to engage the LA facing side 15 of the atrial septum 11 when the IMD 160 is implanted in the heart H, as illustrated. In some cases, the septal portion 178 may include an electrically insulating outer surface. In some instances, the hook portion 180 may be movable between a substantially linear configuration for insertion through the ventricular septum 10 and a curved or hooked configuration (as shown) in which the hook portion 180 positions an LA electrode 182 in contact with the LA facing side 15 of the atrial septum 11. The LA electrode 182 may be electrically connected to control circuitry housed by the housing 162 by one or more conductors extending along the hook portion 180 and septal portion 178. The hook portion 180 may also function to anchor the IMD 160 to the atrial septum 11.

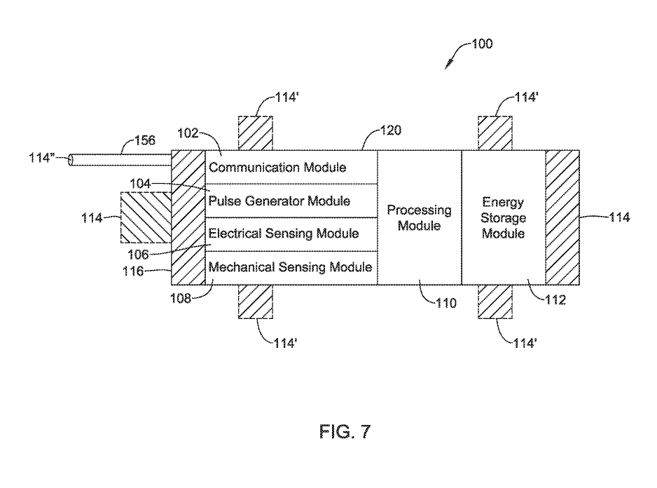

FIG. 7 is a conceptual schematic block diagram of an illustrative IMD, and more specifically a leadless cardiac pacemaker (LCP) that may operate to sense physiological signals and parameters and deliver one or more types of electrical stimulation therapy to the heart of the patient. Example electrical stimulation therapy may include bradycardia pacing, rate responsive pacing therapy, cardiac resynchronization therapy (CRT), anti-tachycardia pacing (ATP) therapy and/or the like. As can be seen in FIG. 7, the LCP 100 may be a compact device with all components housed within the LCP 100 or directly on a housing 120. In some instances, the LCP 100 may include one or more of a communication module 102, a pulse generator module 104, an electrical sensing module 106, a mechanical sensing module 108, a processing module 110, an energy storage module 112, and electrodes 114. The LCP 100 may, for example, be considered as being an example of the IMD 20 (FIGS. 2 and 3), the IMD 50 (FIG. 4) the IMD 60 (FIGS. 5 and 6) and/or the IMD 160 (FIG. 6).

As depicted in FIG. 7, the LCP 100 may include electrodes 114, which can be secured relative to the housing 120 and electrically exposed to tissue and/or blood surrounding the LCP 100. The electrodes 114 may generally conduct electrical signals to and from the LCP 100 and the surrounding tissue and/or blood. Such electrical signals can include communication signals, electrical stimulation pulses, and intrinsic cardiac electrical signals, to name a few. Intrinsic cardiac electrical signals may include electrical signals generated by the heart and may be represented by an electrocardiogram (ECG).

The electrodes 114 may include one or more biocompatible conductive materials such as various metals or alloys that are known to be safe for implantation within a human body. In some instances, the electrodes 114 may be generally disposed on either end of the LCP 100 and may be in electrical communication with one or more of modules the 102, 104, 106, 108, and 110. In embodiments where the electrodes 114 are secured directly to the housing 120, an insulative material may electrically isolate the electrodes 114 from adjacent electrodes, the housing 120, and/or other parts of the LCP 100. In some instances, some or all of the electrodes 114 may be spaced from the housing 120 and may be connected to the housing 120 and/or other components of the LCP 100 through connecting wires. In such instances, the electrodes 114 may be placed on a tail (not shown) that extends out away from the housing 120. As shown in FIG. 7, in some embodiments, the LCP 100 may include electrodes 114′. The electrodes 114′ may be in addition to the electrodes 114, or may replace one or more of the electrodes 114. The electrodes 114′ may be similar to the electrodes 114 except that the electrodes 114′ are disposed on the sides of the LCP 100. In some cases, the electrodes 114′ may increase the number of electrodes by which the LCP 100 may deliver communication signals and/or electrical stimulation pulses, and/or may sense intrinsic cardiac electrical signals, communication signals, and/or electrical stimulation pulses.

The electrodes 114 and/or 114′ may assume any of a variety of sizes and/or shapes, and may be spaced at any of a variety of spacings. For example, the electrodes 114 may have an outer diameter of two to twenty millimeters (mm). In other embodiments, the electrodes 114 and/or 114′ may have a diameter of two, three, five, seven millimeters (mm), or any other suitable diameter, dimension and/or shape. Example lengths for the electrodes 114 and/or 114′ may include, for example, one, three, five, ten millimeters (mm), or any other suitable length. As used herein, the length is a dimension of the electrodes 114 and/or 114′ that extends away from the outer surface of the housing 120. In some instances, at least some of the electrodes 114 and/or 114′ may be spaced from one another by a distance of twenty, thirty, forty, fifty millimeters (mm), or any other suitable spacing. The electrodes 114 and/or 114′ of a single device may have different sizes with respect to each other, and the spacing and/or lengths of the electrodes on the device may or may not be uniform.

In some instances, an LV electrode 114″ may also be provided. The LV electrode 114″ may be supported by an LV electrode support 156 that extends away from the housing 120. In some cases, the LV electrode support 156 is configured to place the LV electrode 114″ within the ventricular septum 10, and in electrical communication with conduction pathways extending through the ventricular septum 10 that control the contraction of the LV. In some instances, the LV electrode support 156 may extend entirely through the ventricular septum 10 in order to place an LV electrode 114″ within the LV and in contact with the LV facing side 14 of the ventricular septum 10.

In the embodiment shown, the communication module 102 may be electrically coupled to two or more of the electrodes 114, 114′ and/or 114″ and may be configured to deliver communication pulses to tissues of the patient for communicating with other devices such as sensors, programmers, other medical devices, and/or the like. Communication signals, as used herein, may be any modulated signal that conveys information to another device, either by itself or in conjunction with one or more other modulated signals. In some embodiments, communication signals may be limited to sub-threshold signals that do not result in capture of the heart yet still convey information. The communication signals may be delivered to another device that is located either external or internal to the patient's body. In some instances, the communication may take the form of distinct communication pulses separated by various amounts of time. In some of these cases, the timing between successive pulses may convey information. The communication module 102 may additionally be configured to sense for communication signals delivered by other devices, which may be located external or internal to the patient's body.

The communication module 102 may communicate to help accomplish one or more desired functions. Some example functions include delivering sensed data, using communicated data for determining occurrences of events such as arrhythmias, coordinating delivery of electrical stimulation therapy, and/or other functions. In some cases, the LCP 100 may use communication signals to communicate raw information, processed information, messages and/or commands, and/or other data. Raw information may include information such as sensed electrical signals (e.g. a sensed ECG), signals gathered from coupled sensors, and the like. In some embodiments, the processed information may include signals that have been filtered using one or more signal processing techniques. Processed information may also include parameters and/or events that are determined by the LCP 100 and/or another device, such as a determined heart rate, timing of determined heartbeats, timing of other determined events, determinations of threshold crossings, expirations of monitored time periods, accelerometer signals, activity level parameters, blood-oxygen parameters, blood pressure parameters, heart sound parameters, and the like. In some cases, processed information may, for example, be provided by a chemical sensor or an optically interfaced sensor. Messages and/or commands may include instructions or the like directing another device to take action, notifications of imminent actions of the sending device, requests for reading from the receiving device, requests for writing data to the receiving device, information messages, and/or other messages commands.

In at least some embodiments, the communication module 102 (or the LCP 100) may further include switching circuitry to selectively connect one or more of the electrodes 114, 114′ and/or 114″ to the communication module 102 in order to select which of the electrodes 114, 114′ and/or 114″ that the communication module 102 delivers communication pulses with. It is contemplated that the communication module 102 may be communicating with other devices via conducted signals, radio frequency (RF) signals, optical signals, acoustic signals, inductive coupling, and/or any other suitable communication methodology. Where the communication module 102 generates electrical communication signals, the communication module 102 may include one or more capacitor elements and/or other charge storage devices to aid in generating and delivering communication signals. In the embodiment shown, the communication module 102 may use energy stored in the energy storage module 112 to generate the communication signals. In at least some examples, the communication module 102 may include a switching circuit that is connected to the energy storage module 112 and, with the switching circuitry, may connect the energy storage module 112 to one or more of the electrodes 114/114′/114″ to generate the communication signals.

As shown in FIG. 7, a pulse generator module 104 may be electrically connected to one or more of the electrodes 114, 114′ and/or 114″. The pulse generator module 104 may be configured to generate electrical stimulation pulses and deliver the electrical stimulation pulses to tissues of a patient via one or more of the electrodes 114, 114′ and/or 114″ in order to effectuate one or more electrical stimulation therapies. Electrical stimulation pulses as used herein are meant to encompass any electrical signals that may be delivered to tissue of a patient for purposes of treatment of any type of disease or abnormality. For example, when used to treat heart disease, the pulse generator module 104 may generate electrical stimulation pacing pulses for capturing the heart of the patient, i.e. causing the heart to contract in response to the delivered electrical stimulation pulse. In some of these cases, the LCP 100 may vary the rate at which the pulse generator module 104 generates the electrical stimulation pulses, for example in rate adaptive pacing. In other embodiments, the electrical stimulation pulses may include defibrillation/cardioversion pulses for shocking the heart out of fibrillation or into a normal heart rhythm. In yet other embodiments, the electrical stimulation pulses may include anti-tachycardia pacing (ATP) pulses. It should be understood that these are just some examples. The pulse generator module 104 may include one or more capacitor elements and/or other charge storage devices to aid in generating and delivering appropriate electrical stimulation pulses. In at least some embodiments, the pulse generator module 104 may use energy stored in the energy storage module 112 to generate the electrical stimulation pulses. In some particular embodiments, the pulse generator module 104 may include a switching circuit that is connected to the energy storage module 112 and may connect the energy storage module 112 to one or more of the electrodes 114/114′/114″ to generate electrical stimulation pulses. In some cases, the pulse generator module 104 may provide pacing pulses to pace the RV of the heart H using electrode 114, and may provide pacing pulses to the LV of the heart H using electrode 114″. In some cases, the pacing pulses generated for pacing the RV of the heart H by the pulse generator module 104 may be offset in time, have a different duration, have a different amplitude and/or have a different shape from the pacing pulses generated by the pulse generator module 104 for pacing the LV of the heart H, if desired.

The LCP 100 may further include an electrical sensing module 106 and a mechanical sensing module 108. The electrical sensing module 106 may be configured to sense intrinsic cardiac electrical signals conducted from the electrodes 114, 114′ and/or 114″ to the electrical sensing module 106. For example, the electrical sensing module 106 may be electrically connected to one or more of the electrodes 114, 114′ and/or 114″ and the electrical sensing module 106 may be configured to receive cardiac electrical signals conducted through the electrodes 114, 114′ and/or 114″ via a sensor amplifier or the like. In some embodiments, the cardiac electrical signals from electrodes 114 and/or 114′ may represent local information from the RV, while the cardiac electrical signals from LV electrode 114″ may represent local information from the LV of the heart H.

The mechanical sensing module 108 may include, or be electrically connected to, various sensors, such as accelerometers, including multi-axis accelerometers such as two- or three-axis accelerometers, gyroscopes, including multi-axis gyroscopes such as two- or three-axis gyroscopes, blood pressure sensors, heart sound sensors, piezoelectric sensors, blood-oxygen sensors, and/or other sensors which measure one or more physiological parameters of the heart and/or patient. Mechanical sensing module 108, when present, may gather signals from the sensors indicative of the various physiological parameters. The electrical sensing module 106 and the mechanical sensing module 108 may both be connected to the processing module 110 and may provide signals representative of the sensed cardiac electrical signals and/or physiological signals to the processing module 110. Although described with respect to FIG. 7 as separate sensing modules, in some embodiments, the electrical sensing module 106 and the mechanical sensing module 108 may be combined into a single module. In at least some examples, the LCP 100 may only include one of the electrical sensing module 106 and the mechanical sensing module 108. In some cases, any combination of the processing module 110, the electrical sensing module 106, the mechanical sensing module 108, the communication module 102, the pulse generator module 104 and/or the energy storage module may be considered a controller of the LCP 100.

The processing module 110 may be configured to direct the operation of the LCP 100 and may, in some embodiments, be termed a controller. For example, the processing module 110 may be configured to receive cardiac electrical signals from the electrical sensing module 106 and/or physiological signals from the mechanical sensing module 108. Based on the received signals, the processing module 110 may determine, for example, occurrences and types of arrhythmias and other determinations such as whether the LCP 100 has become dislodged. The processing module 110 may further receive information from the communication module 102. In some embodiments, the processing module 110 may additionally use such received information to determine occurrences and types of arrhythmias and/or and other determinations such as whether the LCP 100 has become dislodged. In still some additional embodiments, the LCP 100 may use the received information instead of the signals received from the electrical sensing module 106 and/or the mechanical sensing module 108—for instance if the received information is deemed to be more accurate than the signals received from the electrical sensing module 106 and/or the mechanical sensing module 108 or if the electrical sensing module 106 and/or the mechanical sensing module 108 have been disabled or omitted from the LCP 100.

After determining an occurrence of an arrhythmia, the processing module 110 may control the pulse generator module 104 to generate electrical stimulation pulses in accordance with one or more electrical stimulation therapies to treat the determined arrhythmia. For example, the processing module 110 may control the pulse generator module 104 to generate pacing pulses with varying parameters and in different sequences to effectuate one or more electrical stimulation therapies. As one example, in controlling the pulse generator module 104 to deliver bradycardia pacing therapy, the processing module 110 may control the pulse generator module 104 to deliver pacing pulses designed to capture the heart of the patient at a regular interval to help prevent the heart of a patient from falling below a predetermined threshold. In some cases, the rate of pacing may be increased with an increased activity level of the patient (e.g. rate adaptive pacing). For instance, the processing module 110 may monitor one or more physiological parameters of the patient which may indicate a need for an increased heart rate (e.g. due to increased metabolic demand). The processing module 110 may then increase the rate at which the pulse generator module 104 generates electrical stimulation pulses. Adjusting the rate of delivery of the electrical stimulation pulses based on the one or more physiological parameters may extend the battery life of the LCP 100 by only requiring higher rates of delivery of electrical stimulation pulses when the physiological parameters indicate there is a need for increased cardiac output. Additionally, adjusting the rate of delivery of the electrical stimulation pulses may increase a comfort level of the patient by more closely matching the rate of delivery of electrical stimulation pulses with the cardiac output need of the patient.

For ATP therapy, the processing module 110 may control the pulse generator module 104 to deliver pacing pulses at a rate faster than an intrinsic heart rate of a patient in attempt to force the heart to beat in response to the delivered pacing pulses rather than in response to intrinsic cardiac electrical signals. Once the heart is following the pacing pulses, the processing module 110 may control the pulse generator module 104 to reduce the rate of delivered pacing pulses down to a safer level. In CRT, the processing module 110 may control the pulse generator module 104 to deliver pacing pulses in coordination with another device to cause the heart to contract more efficiently. In cases where the pulse generator module 104 is capable of generating defibrillation and/or cardioversion pulses for defibrillation/cardioversion therapy, the processing module 110 may control the pulse generator module 104 to generate such defibrillation and/or cardioversion pulses. In some cases, the processing module 110 may control the pulse generator module 104 to generate electrical stimulation pulses to provide electrical stimulation therapies different than those examples described above.

Aside from controlling the pulse generator module 104 to generate different types of electrical stimulation pulses and in different sequences, in some embodiments, the processing module 110 may also control the pulse generator module 104 to generate the various electrical stimulation pulses with varying pulse parameters. For example, each electrical stimulation pulse may have a pulse width and a pulse amplitude. The processing module 110 may control the pulse generator module 104 to generate the various electrical stimulation pulses with specific pulse widths and pulse amplitudes. For example, the processing module 110 may cause the pulse generator module 104 to adjust the pulse width and/or the pulse amplitude of electrical stimulation pulses if the electrical stimulation pulses are not effectively capturing the heart (e.g. RV or LV capture). Such control of the specific parameters of the various electrical stimulation pulses may help the LCP 100 provide more effective delivery of electrical stimulation therapy.

In some embodiments, the processing module 110 may further control the communication module 102 to send information to other devices. For example, the processing module 110 may control the communication module 102 to generate one or more communication signals for communicating with other devices of a system of devices. For instance, the processing module 110 may control the communication module 102 to generate communication signals in particular pulse sequences, where the specific sequences convey different information. The communication module 102 may also receive communication signals for potential action by the processing module 110.

In further embodiments, the processing module 110 may control switching circuitry by which the communication module 102 and the pulse generator module 104 deliver communication signals and/or electrical stimulation pulses to tissue of the patient. As described above, both the communication module 102 and the pulse generator module 104 may include circuitry for connecting one or more of the electrodes 114, 114′ and/or 114″ to the communication module 102 and/or the pulse generator module 104 so those modules may deliver the communication signals and electrical stimulation pulses to tissue of the patient. The specific combination of one or more electrodes by which the communication module 102 and/or the pulse generator module 104 deliver communication signals and electrical stimulation pulses may influence the reception of communication signals and/or the effectiveness of electrical stimulation pulses. Although it was described that each of the communication module 102 and the pulse generator module 104 may include switching circuitry, in some embodiments, the LCP 100 may have a single switching module connected to the communication module 102, the pulse generator module 104, and the electrodes 114, 114′ and/or 114″. In such embodiments, processing module 110 may control the switching module to connect the modules 102/104 and the electrodes 114/114′/114″ as appropriate. In some cases, the LV electrode 114″ may also be coupled to the switching module and may be used for communication.

In some embodiments, the processing module 110 may include a pre-programmed chip, such as a very-large-scale integration (VLSI) chip or an application specific integrated circuit (ASIC). In such embodiments, the chip may be pre-programmed with control logic in order to control the operation of the LCP 100. By using a pre-programmed chip, the processing module 110 may use less power than other programmable circuits while able to maintain basic functionality, thereby potentially increasing the battery life of the LCP 100. In other instances, the processing module 110 may include a programmable microprocessor or the like. Such a programmable microprocessor may allow a user to adjust the control logic of the LCP 100 after manufacture, thereby allowing for greater flexibility of the LCP 100 than when using a pre-programmed chip. In still other embodiments, the processing module 110 may not be a single component. For example, the processing module 110 may include multiple components positioned at disparate locations within the LCP 100 in order to perform the various described functions. For example, certain functions may be performed in one component of the processing module 110, while other functions are performed in a separate component of the processing module 110.

The processing module 110, in additional embodiments, may include a memory circuit and the processing module 110 may store information on and read information from the memory circuit. In other embodiments, the LCP 100 may include a separate memory circuit (not shown) that is in communication with the processing module 110, such that the processing module 110 may read and write information to and from the separate memory circuit. The memory circuit, whether part of the processing module 110 or separate from the processing module 110, may be volatile memory, non-volatile memory, or a combination of volatile memory and non-volatile memory.

The energy storage module 112 may provide a power source to the LCP 100 for its operations. In some embodiments, the energy storage module 112 may be a non-rechargeable lithium-based battery. In other embodiments, the non-rechargeable battery may be made from other suitable materials. In some embodiments, the energy storage module 112 may be considered to be a rechargeable power supply, such as but not limited to, a rechargeable battery. In still other embodiments, the energy storage module 112 may include other types of energy storage devices such as capacitors or super capacitors. In some cases, as will be discussed, the energy storage module 112 may include a rechargeable primary battery and a non-rechargeable secondary battery. In some cases, the primary battery and the second battery, if present, may both be rechargeable.

To implant the LCP 100 inside a patient's body, an operator (e.g., a physician, clinician, etc.), may fix the LCP 100 to the cardiac tissue of the patient's heart. To facilitate fixation, the LCP 100 may include one or more anchors 116. The one or more anchors 116 are shown schematically in FIG. 7. The one or more anchors 116 may include any number of fixation or anchoring mechanisms. For example, one or more anchors 116 may include one or more pins, staples, threads, screws, helix, tines, and/or the like. In some embodiments, although not shown, one or more anchors 116 may include threads on its external surface that may run along at least a partial length of an anchor member. The threads may provide friction between the cardiac tissue and the anchor to help fix the anchor member within the cardiac tissue. In some cases, the one or more anchors 116 may include an anchor member that has a cork-screw shape that can be screwed into the cardiac tissue. In other embodiments, the anchor 116 may include other structures such as barbs, spikes, or the like to facilitate engagement with the surrounding cardiac tissue. In some cases, the LV electrode support 156 may anchor the LCP 100, such as described above with respect to FIG. 5.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments.

What is claimed is:

1. An implantable medical device (IMD) configured for deployment at a ventricular septum of a patient's heart, the ventricular septum of the patient's heart having a right ventricle (RV) facing side and a left ventricle (LV) facing side, the IMD comprising:
a housing configured to be positioned at least in part in the right ventricle (RV) of the patient's heart proximate the RV facing side of the ventricular septum once the IMD is implanted in the patient's heart;
a power source disposed within the housing;
circuitry disposed within the housing and operatively coupled to the power source;
a first RV electrode fixed relative to the housing and positioned to be proximate the RV facing side of the ventricular septum once the IMD is implanted, the RV electrode operatively coupled with the circuitry in the housing;
a second RV electrode fixed relative to the housing and spaced from the first RV electrode, the second RV electrode operatively coupled with the circuitry in the housing;
an LV electrode support extending away from the housing into the ventricular septum toward the LV facing side of the ventricular septum;
an LV electrode supported by the LV electrode support and spaced from the housing, the LV electrode operatively coupled with the circuitry in the housing; and
the circuitry configured to pace the RV of the patient's heart using the first RV electrode and/or the second RV electrode and to pace the LV of the patient's heart using the LV electrode.

2. The IMD of claim 1, wherein the first RV electrode and/or the second RV electrode are disposed on an outer surface of the housing.

3. The IMD of claim 1, wherein the LV electrode support and the LV electrode are configured to position the LV electrode within the ventricular septum.

4. The IMD of claim 1, wherein the LV electrode support and the LV electrode are configured to position the LV electrode in contact with the LV facing side of the ventricular septum.

5. The IMD of claim 1, further comprising:
another LV electrode support extending away from the housing into the ventricular septum toward the LV facing side of the ventricular septum; and
another LV electrode supported by the another LV electrode support, the another LV electrode operatively coupled with the circuitry in the housing.

6. The IMD of claim 1, wherein the LV electrode support is configured to extend far enough into the ventricular septum to position the LV electrode for successful capture of the LV of the patient's heart but not all the way through the ventricular septum.

7. The IMD of claim 1, wherein the IMD comprises a dual chamber leadless cardiac pacemaker (LCP).

8. The IMD of claim 1, wherein the power source comprises a battery.

9. The IMD of claim 1, wherein the power source comprises a rechargeable power source.

10. An implantable medical device (IMD) configured for deployment at a ventricular septum of a patient's heart, the ventricular septum of the patient's heart having a right ventricle (RV) facing side defining part of the right ventricle (RV) of the patient's heart and a left ventricle facing side defining part of the left ventricle (LV) of the patient's heart, the IMD comprising:
a housing configured to be positioned at least in part in the right ventricle of the patient's heart proximate the right ventricle facing side of the ventricular septum once the IMD is implanted;
a power source disposed within the housing;
circuitry disposed within the housing and operatively coupled to the power source;
an RV electrode fixed relative to the housing and positioned to engage the right ventricle facing side of the ventricular septum once the IMD is implanted, the RV electrode is operatively coupled with the circuitry;
an LV electrode assembly including:
a septal portion configured to extend away from the housing and through the ventricular septum;
a hook portion extending distally from the septal portion and configured to engage the LV facing side of the ventricular septum once the IMD is implanted;
an LV electrode supported by the hook portion to engage the left ventricle facing side of the ventricular septum once the IMD is implanted; and
the circuitry configured to pace the RV of the patient's heart using the RV electrode and to pace the LV of the patient's heart using the LV electrode.

11. The IMD of claim 10, wherein the LV electrode assembly extends distally from a distal end of the housing and has an effective length sufficient to place the hook portion in contact with the LV facing side of the ventricular septum.

12. The IMD of claim 11, wherein the septal portion of the LV electrode assembly has a length of up to about 2 centimeters.

13. The IMD of claim 10, wherein the LV electrode assembly is configured to anchor the IMD in place relative to the ventricular septum once the IMD is implanted.

14. The IMD of claim 10, wherein the septal portion of the LV electrode assembly includes an electrically insulating outer surface.

15. The IMD of claim 10, wherein the hook portion of the LV electrode assembly is movable between a substantially linear configuration for insertion through the ventricular septum and a curved configuration in which the hook portion positions the LV electrode in contact with the LV facing side of the ventricular septum.

16. The IMD of claim 10, wherein the circuitry is further configured to sense cardiac electrical activity using the RV electrode and the LV electrode.

17. The IMD of claim 10, wherein the power source comprises a battery.

18. The IMD of claim 10, wherein the power source comprises a rechargeable power source.

19. An implantable medical device (IMD) configured for deployment at a ventricular septum of a patient's heart, the ventricular septum of the patient's heart having a right ventricle (RV) facing side defining part of the right ventricle (RV) of the patient's heart and a left ventricle facing side defining part of the left ventricle (LV) of the patient's heart, the IMD comprising:
a housing configured to be positioned at least in part in the right ventricle of the patient's heart proximate the right ventricle facing side of the ventricular septum once the IMD is implanted;
a power source disposed within the housing;
circuitry disposed within the housing and operatively coupled to the power source;
an RV electrode fixed relative to the main housing and positioned to engage the right ventricle facing side of the ventricular septum once the IMD is implanted, the RV electrode operatively coupled with the circuitry;
an LV electrode support configured to extend from the housing into the ventricular septum;
an LV electrode supported by the LV electrode support, the LV electrode operatively coupled with the circuitry; and
the circuitry configured to pace the patient's heart using the RV electrode and the LV electrode.

20. The IMD of claim 19, wherein the LV electrode support is configured to extend far enough into the ventricular septum to position the LV electrode for successful capture of the LV of the patient's heart but not all the way through the ventricular septum.
